(19) United States
(12) Patent Application Publication
(10) Pub. No.: US 2022/0401596 A1
(43) Pub. Date: Dec. 22, 2022

(54) MODULAR DISINFECTION VEHICLE

(71) Applicant: Shenzhen Guanke Technologies Co., Ltd, Shenzhen (CN)

(72) Inventors: Qing Lan, Shenzhen (CN); Tianlong Dai, Shenzhen (CN); Shoubao Chen, Shenzhen (CN); Ligen Liu, Shenzhen (CN); Minggui Wang, Shenzhen (CN)

(73) Assignee: Shenzhen Guanke Technologies Co., Ltd, Shenzhen (CN)

(21) Appl. No.: 17/411,469

(22) Filed: Aug. 25, 2021

(30) Foreign Application Priority Data
Jun. 21, 2021 (CN) 2021213864888

(51) Int. Cl. A61L 2/10; A61L 9/20

(52) U.S. Cl. CPC A61L 2/10; A61L 9/20; A61L 2202/11; A61L 2202/16; A61L 2202/14; A61L 2209/12; A61L 2209/11; A61L 2209/15

(57) ABSTRACT

A modular disinfection vehicle. The disinfection component is designed into an independent extensible structure, where there is a light source module between the upper end and the lower end of the disinfection component, the upper end of the disinfection component is the first fixing part, the lower end of the disinfection component is the second fixing part, the upper end of the disinfection component is of a dismountable structure, so that the light source module can be replaced and maintained conveniently; when the quantity of the light source modules needs to be increased or decreased, only components and parts at the upper end or/and the lower end of the disinfection component need to be replaced, there is no need to significantly improve the overall structure of the disinfection vehicle, so that the quantity of light source modules of the disinfection vehicle can be increased or decreased conveniently.

### FIELD

This invention relates to the technical field of disinfection equipment, particularly to a modular disinfection vehicle.

### BACKGROUND

The UV sterilization and disinfection light mainly includes two working modes: direct illumination disinfection and ventilation disinfection. Direct illumination disinfection light refers to the light which directly exposes UV light in an open environment for sterilization and disinfection; it can be used for disinfection of object surface and air, its disinfection efficiency is high, but the safety performance is poor, so people need to keep themselves away from the light to ensure the safety. Ventilation disinfection light refers to the light which inhales air into the enclosed light cavity for UV disinfection and then exhausts sterilized air out of the light, so that air disinfection and ventilation is finished; it is mostly used for air disinfection, during disinfection, people do not need to leave the disinfection environment, UV inside the light will not cause personal injuries arising from exposure of UV light outside the light, and with high safety performance, it can realize co-existence of human and the light.

Most UV disinfection vehicles adopt direct illumination, and the specifications of disinfection vehicles are relatively unitary, their structures are not suitable for increasing or decreasing the quantity of UV light source modules and will affect series extension and use of disinfection vehicles. In addition, the UV light source inside the disinfection vehicles cannot be repaired or replaced conveniently, because their service life will be affected.

The foregoing content is only used for assisting in understanding the technical solution of this invention, but does not mean the acknowledgement of that the above content is the current technology.

### SUMMARY

The main objective of the present invention is to provide a modular disinfection vehicle to solve technical problems of existing disinfection vehicles like poor safety performance, unitary specification, structure unsuitable for the increase or the decrease of the quantity of UV light source modules, affecting series extension and use of disinfection vehicles and not good for repair or replacement of UV light source modules, etc.

The technical problem solving thinking of the invention is as follows:

On one aspect, the UV light source module of the disinfection vehicle is provided on the independent disinfection component which can be lifted and lowered, when there is no person in the sterilization environment, the light source module will be lifted to carry out direct illumination disinfection; when there is person in the sterilization environment, the light source module retracts into the vehicle body cavity and ventilation disinfection is enabled to improve the use safety of the product. These switchable working mode improves the safety, environment adaptability and sterilization and disinfection efficiency of the disinfection vehicle.

On the other hand, the disinfection component is designed into an independent extensible structure, the light source module is provided between the upper end and the lower end of the disinfection component, the upper end of the disinfection component is the first fixing part, the lower end of the disinfection component is the second fixing part, the upper end of the disinfection component is a dismountable structure which facilitates replacement and maintenance of the light source module. And, the light source module is provided in the dismountable disinfection component in a centralized way. When the quantity of the light source module needs to increase or decrease, only corresponding parts at the upper end or/and the lower end of the disinfection component need to be replaced, that is, only the first fixing part and/or the second fixing part second fixing part in this invention needs to be replaced, and the overall structure of the disinfection vehicle does not need to be improved significantly, which facilitates increase or decrease of the quantity of light source modules of the disinfection vehicle and realize specification derivation and series extension of disinfection vehicles. The disinfection component is provided with a (or several) support pillar/s which can improve the structure intensity of the disinfection component and play a leading and guiding role when the light source module lifts and retracts to make the disinfection vehicle system more stable. The motor drives the lead screw to finish lifting and lowering of the disinfection component, the control system controls lifting and lowering of the disinfection component and working of the UV light source module, including enabling, disenabling, timing, etc.

To realize the above subjective of the present invention, this invention adopts the following technical solution:

a modular disinfection vehicle used for sterilization and disinfection of air or the object surface, characterized in comprising:

a vehicle body with the first accommodating cavity and at least an avoiding hole connecting to the first accommodating cavity and provided on the top of the vehicle body;

a disinfection component, characterized in comprising the first fixing part, the second fixing part, at least a support pillar and at least a light source module, the first fixing part and the second fixing part provided on each end of the support pillar respectively, the second fixing part provided in the first accommodating cavity, the light source module and the support pillar pass through the avoiding hole in a flexible way; the light source module installed on the first fixing part and/or the second fixing part in a dismountable way;

a lifting mechanism, provided in the first accommodating cavity and connecting to the second fixing part to drive the disinfection component to stretch out of and retract to the vehicle body; and

a control system, used for controlling enabling and disenabling of the light source module and controlling the lifting mechanism to drive the disinfection component to stretch out of and retract to the vehicle body in the vertical direction.

The advantages and positive effect of the modular disinfection vehicle in this invention include:

(1) The light source module is provided in the independent disinfection component which can be lifted and lowered, enabling the disinfection vehicle to have two switchable working modes, improve the safety, environment adaptability and sterilization and disinfection of the disinfection vehicle and realize co-existence of human and the vehicle during working of the disinfection vehicle;

(2) The light source module is provided between the first fixing part and the second fixing part in a dismountable way, facilitating replacement and repair of the light source module;

(3) The light source module is provided in the dismountable disinfection component in a centralized way. When the quantity of the light source module needs to increase or decrease, only the corresponding first fixing part and/or the second fixing part need to be replaced, and the overall structure of the disinfection vehicle does not need to be improved significantly, which facilitates increase or decrease of the quantity of light source modules of the disinfection vehicle and realize specification derivation and series extension of disinfection vehicles;

(4) The disinfection component is provided with a (or several) support pillar/s which can improve the structure intensity of the disinfection component and play a leading and guiding role when the light source module lifts and retracts to make the disinfection vehicle system more stable.

### BRIEF DESCRIPTION OF THE DRAWINGS

FIG. 1 is a schematic diagram showing the structure of an example of the modular disinfection vehicle of the present invention;

FIG. 2 is the schematic diagram of enlargement of position A in FIG. 1;

FIG. 3 is the schematic diagram of the cross section of the modular disinfection vehicle;

FIG. 4 is the schematic diagram of enlargement of position B in FIG. 3;

FIG. 5 is the schematic diagram of enlargement of position C in FIG. 3;

FIG. 6 is the breakdown diagram of the modular disinfection vehicle containing a base.

FIG. 7 is the schematic diagram of the disinfection component of the modular disinfection vehicle.

FIG. 8 is the schematic diagram of the vehicle body of the modular disinfection vehicle.

FIG. 9 is the breakdown diagram of the handle of the modular disinfection vehicle.

FIG. 10 is the schematic diagram of the ventilation component of the modular disinfection vehicle.

FIG. 11 is the schematic diagram of the first shielding part of the modular disinfection vehicle.

FIG. 12 is the mounting diagram of the sensing device of the modular disinfection vehicle.

FIG. 13 is the schematic diagram of the inside of the modular disinfection vehicle.

FIG. 14 is the identification diagram of keys of the modular disinfection vehicle.

The shape, dimension, proportion or position relationship of parts of the product in drawings may be real data of embodiments and they are under protection of this invention.

### DETAILED DESCRIPTION

The technical solutions of the embodiments in this present invention will be described in an explicit and intact manner below with reference to the drawings. It is obvious that the specific embodiments described herein are merely part but not all the embodiments of this present invention. All other embodiments obtained by technicians in this field without inventive work based on the embodiments in this present invention are within the protection scope of this present invention.

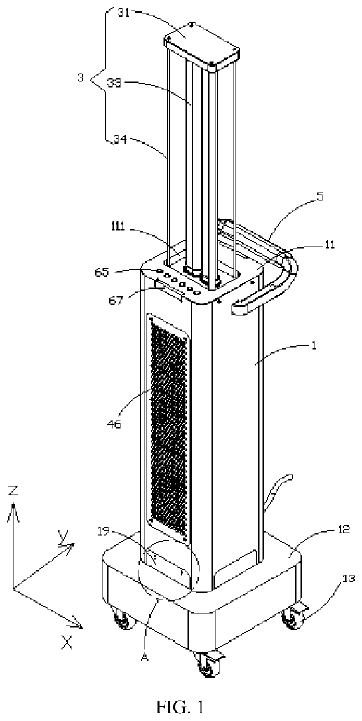

In one example of the present invention as shown in FIG. 1 and FIG. 3, vehicle body 1 possesses the first accommodating cavity 18 and at least one avoiding hole 16 connecting to the first accommodating cavity 18, the avoiding hole 16 is provided on the top of the vehicle body 1, the disinfection component 3 comprises the first fixing part 31, at least one support pillar 34 and at least one light source module 33, the first fixing part 31 and the second fixing part 32 are provided on each end of the support pillar 34 respectively, the second fixing part second fixing part 32 is provided in the first accommodating cavity 18, the light source module 33 and the support pillar 34 pass through the avoiding hole 16 in a flexible way; the light source module 33 is installed onto the first fixing part 31 and/or the second fixing part 32 in a dismountable way, and the lifting mechanism 2 connects to the second fixing part 32 to drive the disinfection component 3 to stretch out and retract to the vehicle body 1, the control system 6 is used for controlling enabling and disenabling of the light source module 33 and controlling the lifting mechanism 2 to drive the disinfection component 3 to stretch out of and retract to the vehicle body 1 in the vertical direction.

In the solution of this invention, the light source module 33 of the disinfection vehicle is provided in the independent disinfection component 3 which can be lifted and lowered, enabling the disinfection vehicle to have two switchable working modes. Under the first working mode, the lifting component controls the disinfection component 3 to stretch out of the outer side of the vehicle body 1 to carry out direct illumination disinfection of the surrounding environment; Under the second working mode, the lifting component controls the disinfection component 3 to retracts to the first accommodating cavity 18 to sterilize air in the first accommodating cavity 18 to guarantee the safety, environment adaptability and sterilization and disinfection efficiency of the disinfection vehicle and realize co-existence of human and machine during working of the disinfection vehicle.

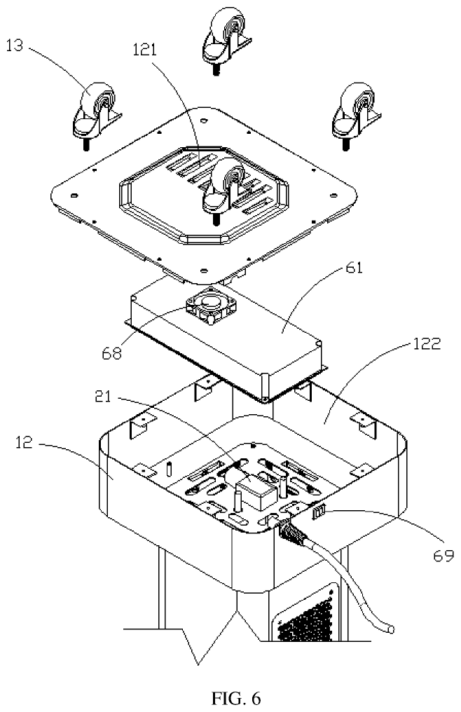

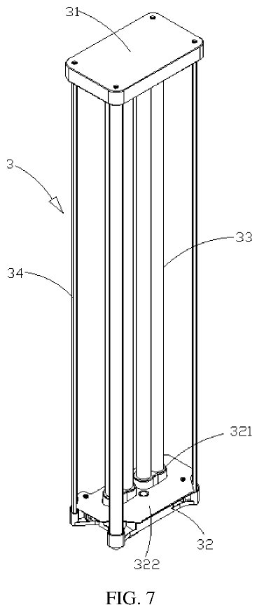

The dismountable light source module 33 can be provided on the first fixing part 31 or the second fixing part 32 or between them. As shown in FIG. 3 and FIG. 7, the first fixing part 31 is provided with the first installation position 311, the second fixing part 32 is provided with the second installation position 321, both sides of the light source module 33 are installed respectively on the first installation position 311 and the second installation position 321. The first fixing part 31 and the support pillar 34 are connected in a dismountable way. After the first fixing part 31 and the support pillar 34 are dismounted, the light source module 33 can be separated from the first installation position 311 and the second installation position 321 to finish disassembly and replacement of the light source module 33.

In the embodiment as shown in FIG. 3, there are two light source modules 33. When the quantity of light source modules 33 is increased or decreased, only the first fixing part 31 and/or second fixing part 32 need/s to be replaced, the overall structure of the disinfection vehicle does not need to be improved significantly, which facilitates increase or decrease of the quantity of light source modules 33 of the disinfection vehicle, realizes specification derivation and series extension of disinfection vehicles and facilitates repair and replacement of the light source module 33. The support pillar 34 can improve the structure intensity of the disinfection component 3 and play a leading and guiding role when the light source module 33 lifts and retracts to make the disinfection vehicle system more stable.

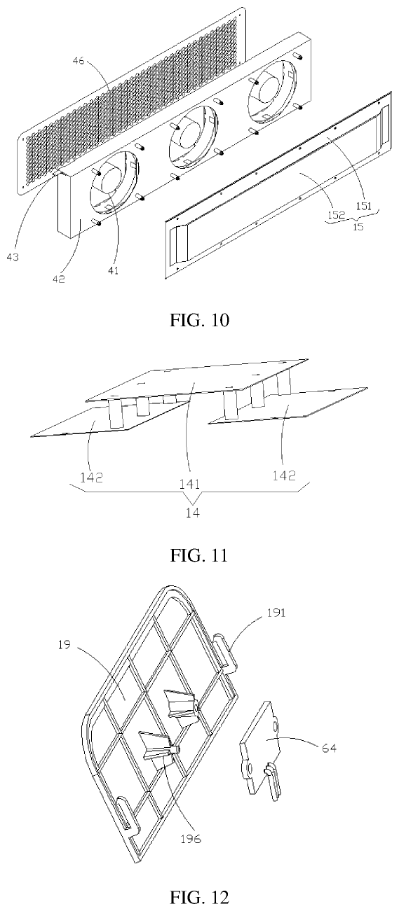

According to FIG. 1-14, the disinfection vehicle comprises the vehicle body 1, a disinfection component 3, a lifting mechanism 2, a ventilation component 4, a control system 6, a handle 5 and lead wire 7. In one example of the present invention the z-axis direction in FIG. 1 is defined as the vertical direction, and the lifting mechanism 2 drives the disinfection component 3 to lift and lower in the vertical direction.

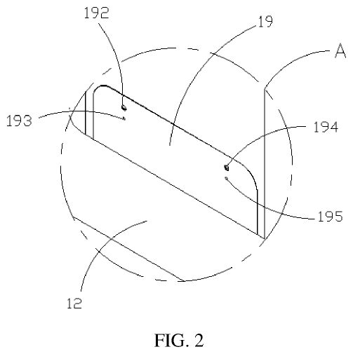

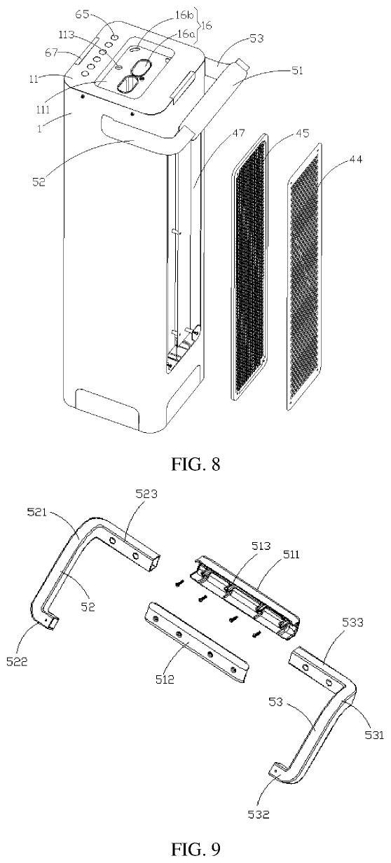

As shown in FIG. 2 and FIG. 8, the top of the vehicle body 1 is provided with the top cover 11, the bottom of the vehicle body 1 is provided with a base 12, the bottom of the base 12 is provided with wheels 13 for facilitating movement of the disinfection vehicle. The vehicle body 1 possesses the first accommodating cavity 18, the top cover 11 and other parts of the vehicle body 1 form the second accommodating cavity 112 in the way of enclosing, the second accommodating cavity 112 is used for accommodating electronic elements needed by the disinfection vehicle, and there is the third accommodating cavity 122 connecting to the first accommodating cavity 18 inside the base 12.

The top cover 11 is provided with at least one avoiding hole 16 connecting to first accommodating cavity 18. In one example of the present invention according to the functions of each avoiding hole 16, there is the first avoiding hole 16a, the second avoiding hole 16b and the third avoiding hole 113. As shown in FIG. 3 and FIG. 7, the light source module 33 passes through the first avoiding hole 16a in a flexible way, and the support pillar 34 passes through the second avoiding hole 16b in a flexible way.

In one example of the present invention the middle of the outer side of the top cover 11 is provided with a groove 111, the horizontal projection plane of the first fixing part 31 is rectangular on the whole, which facilitates accommodation of 1-4 light source modules 33, and the horizontal projection plane of the groove 111 is rectangular on the whole. The projection plane is rectangular on the whole, which means that the shape of the projection plane is approximately rectangular, such as rectangle, rectangle provided with a chamfer or oval. The first avoiding hole 16a, the second avoiding hole 16b and the third avoiding hole 113 are provided at the bottom of the groove 111 respectively. When the disinfection component 3 retracts to the vehicle body 1, the first fixing part 31 can be placed inside the groove 111.

Optionally, there are four support pillars 34 provided on the outer side of the disinfection component 3. The support pillar 34 is of a profile structure and the outer contour of the cross section is non-circular, avoiding that the support pillar 34 rotates relative to the first fixing part 31 and/or the second fixing part 32. There are four second avoiding holes 16b which enable each support pillar 34 to pass through different second avoiding hole 16b to ensure stable and smooth movement of the disinfection component 3.

The solution of this invention can also be understood like this: the support pillar 34 and the light source module 33 share one avoiding hole 16, which can reduce the quantity of holes on the top cover 11, reduce the processing technology of the top cover 11 and improve the assembly efficiency of the disinfection component 3.

As shown in FIG. 3 and FIG. 5, the lifting mechanism 2 comprises a motor 21, a nut 22 and a lead screw 23. The motor 21 connects to the lead screw 23, motor 21 is installed inside the third accommodating cavity 122 of the base 12, lead screw 23 is provided vertically inside the first accommodating cavity 18, the second fixing part 32 is provided with a vertical third through-hole which is in the middle of the second fixing part 32 to ensure the evenness of load bearing of the lead screw 23. The vehicle body 1 is provided with a positioning hole 114. the lead screw 23 is provided with a shaft sleeve 24 provided inside the positioning hole 114. The support pillar 34 match with the second avoiding hole 16b in a slide way, there is definitely a clearance between the support pillar 34 and the second avoiding hole 16b, the thin and long lead screw 23 will generate great eccentricity when the arm of force is too long, so there will be a problem that the disinfection component 3 fails to lift because of the great friction between the inclined support pillar 34 and the second avoiding hole 16b, but the design of the shaft sleeve 24 and the positioning hole 114 can avoid inclination or jitter of the disinfection component 3 during lifting, so that the stability of the disinfection component 3 can be improved. The shaft sleeve 24 of a sleeve structure is provided at the end of the lead screw. It is made from nylon or other wear-resistant materials. Compared with ordinary rolling bearings, shaft sleeve 24 is characterized by easy processing, simple installation and low cost. The outer side of the vehicle body 1 is provided with an oil leakage hole 115 corresponding to the positioning hole 114. In this embodiment, the oil leakage hole 115 is provided on the vehicle body 1, the oil leakage hole 115 connects to the positioning hole 114, lubricating oil can be applied to lead screw 23 via the oil leakage hole 115 to ensure that the nut 22 can smoothly move along the lead screw 23.

In one example of the present invention rotating motor 21 drives the lead screw 23 to rotate to make nut 22 move along the lead screw 23. By controlling the rotation direction of the rotating shaft of motor 21, the nut 22 can move up and down along the lead screw 23 to make the light source module 33 stretch out of or retract to vehicle body 1. Since the support pillar 34 passes through the second avoiding hole 16b, the second avoiding hole 16b can control the support pillar 34 to only move up and down, so that the disinfection component 3 can lift and lower smoothly and stably. Additionally, the lifting mechanism is of a concise and compact structure, the disinfection component 3 is very light, so small-power motor 21 and thin lead screw 23 can be used. In this embodiment, the diameter of the lead screw 23 is 6-10 mm (8 mm is the optimal). The technician can also set the diameter of the lead screw 23 according to the size and specification of the disinfection vehicle.

The motor 21 is provided with a gear mechanism which can reduce the rotation speed of the motor 21 and provide bigger output torque. The motor 21 connects to the lead screw 23 via the gear mechanism.

As shown in FIGS. 1-3, 5, 8 and 10-11, the ventilation component 4 is provided inside the first accommodating cavity, vehicle body 1 is provided with the first ventilation hole 47 and the second ventilation hole 48 connecting to the first accommodating cavity 18, the ventilation component 4 is provided in the second ventilation hole 48, air passes through the first ventilation hole 47 and enters into the first accommodating cavity 18 where sterilization and disinfection is finished, and then air is exhausted out of the vehicle body 1 via the second ventilation hole 48, the vehicle body 1 is provided with a shielding device corresponding to the first ventilation hole 47 and the second ventilation hole 48 respectively, the shielding device is used for preventing UV light emitted by light source module 33 inside the first accommodating cavity 18 going out of the vehicle body 1. Furthermore, according to FIG. 5 and FIG. 7, the second fixing part 32 is provided with the third shielding part 322 which is provided on the top of the second fixing part 32, the third shielding part 322 is used for the shielding avoiding hole 16, after the disinfection component 3 stretches out of the vehicle body 1, the third shielding part 322 can prevent UV light from entering the first accommodating cavity 18 via the avoiding hole 16, which can protect the lower part of the light source module 33 and protect parts (plastic parts, lead wire 7) inside the vehicle body 1 from damage caused by UV light.

In one example of the present invention one shielding device can be the first shielding part 14 which comprises one first board 141 and two second boards 142 provided at intervals. The first board 141 is provided above the clearance of two second boards 142 in the stack-up way, enabling the UV light emitted by the light source module 33 to not go out of the vehicle body 1 in the direction vertical to the first board 141 and second boards 142. The other shielding device is the second shielding part 15 which comprises the fourth board 151 and the fifth board 152. The middle part of the fourth board 151 is provided with a ventilation hole. The fifth board 152 is provided at the ventilation hole of the fourth board 151 at intervals in a stack-up way. The fifth board 152 covers the ventilation hole of the fourth board 151, preventing the UV light from being emitted out of the vehicle body via the ventilation hole.

Optionally, the first board 141 and the second boards 142 are in parallel. There is the third mounting column between the first board 141 and the second boards 142. There may be several mounting columns. One end of the third mounting column connects to the first board 141, the other end of the third mounting column connects to the second board 142 to facilitate processing of the first shielding part 14 and increase the ventilation hole of the first shielding part 14.

The ventilation component 4 comprises a fan housing 42 and at least one first fan 41. The fan housing 42 is provided with the fourth ventilation hole corresponding to the first fan 41. The fan housing 42 is against the inner wall of the vehicle body 1, so that air in the first accommodating cavity 18 can be exhausted out of the vehicle body 1 via the second ventilation hole 48 after passing through the fourth ventilation hole, the first fan 41 can generate air faster and blower sterilized air further. The fan housing 42 provided on the second shielding part 15 faces one side of the second ventilation hole 48. The second shielding part 15 and the fan housing 42 can prevent the fan from the damage of the light emitted by the light source module 33. In one example of the present invention as shown in FIG. 10, there are three first fans 41, the fan housing 42 is provided with three fourth ventilation holes arranged along the length of the second ventilation hole, each fourth ventilation hole is provided with one first fan 41, to facilitate access of the first fan 41 to the external power supply, the fan housing 42 is provided with a wiring board 43, the first fan 41 connects to the wiring board 43 electrically, and the wiring board 43 extends to pass through the fourth ventilation hole and then stretch out of the housing.

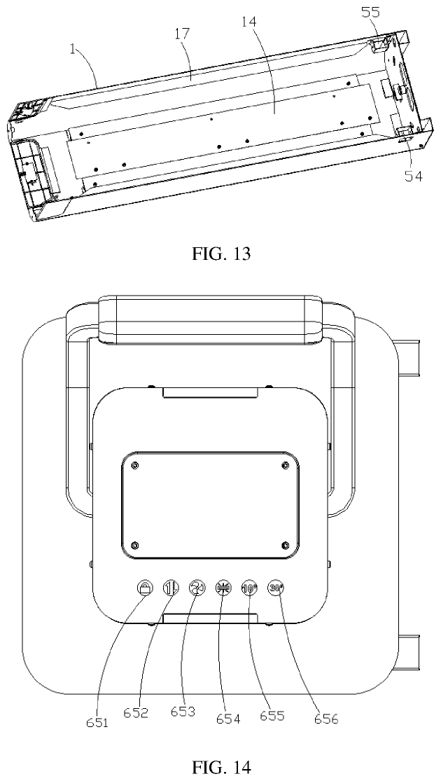

In one example of the present invention the first ventilation hole 47 and the second ventilation hole 48 are disposed opposite each other on two sides of the vehicle body 1, so that the incoming air of the vehicle body 1 will not affect its outgoing air and the disinfection efficiency of the disinfection vehicle is improved. As shown in FIG. 13, the vehicle body 1 is provided with two rib slabs 17 on the lateral side between the first ventilation hole 47 and the second ventilation hole 48, rib slabs 17 is provided corresponding to the light source module 33, when air flows from the first ventilation hole 47 to the second ventilation hole 48, since the impediment of the rib slabs 17, air will be closer to the light source module 33 and the sterilization efficiency will be improved.

The first ventilation hole 47 is provided with a filter screen 45 in a dismountable way, the filter screen 45 filters the air which flows into the first accommodating cavity 18 to ensure the cleanliness inside the vehicle body 1, and the filter screen 45 is dismountable in the way of screw fastening or clamping. In one example of the present invention the first ventilation hole 47 is provided with the first protective net 44, and the second ventilation hole 48 is provided with the second protective net 46 to improve the use safety of the disinfection vehicle.

The control system 6 also comprises the first circuit board 66 which is provided inside the second accommodating cavity 112 and connects to at least one key 65, the key 65 stretches out of the top of the vehicle body 1, the top cover 11 is provided with a hole for stretching of the key 65, the key 65 comprises a keycap and a button, the keycap covers the key, the keycap stretches out of the top cover 11 from the hole, key 65 can be used for controlling the lifting mechanism 2 to stretch out or retract, controlling the disinfection component 3 to be enabled or disenabled, or controlling the working time of the light source module 33. As shown in FIG. 14, there are six keys 65, i.e. child lock key 651, lift key 652, fan key 653, disinfection key 654, the first timing key 655 and the second timing key 656. Wherein, the child lock key 651 is used for locking other keys and preventing misoperation; the lift key 652 is used for controlling the disinfection component 3 to stretch out of or retract to the vehicle body 1; the fan key 653 is used for switching the gear of the first fan 41; The disinfection key 654 is used for controlling the enabling and disenabling of the light source module 33; the first timing key 655 and the second timing key 656 are used for setting the working time of the light source module 33. For example, they can be used for setting the sterilization time to be 10 min or 30 min. The disinfection vehicle can be operated via several keys 65 or remote control.

The control system 6 also comprises a working indicator 67 provided on the top of the vehicle body 1 and used for displaying the working state of the light source module 33. The working indicator 67 comprises an indicator light source and a transparent cover. The indicator light source is provided on the first circuit board 66, and the transparent cover is provided on the intersection of the top and lateral side of the vehicle body 1 corresponding to the indicator light source. The transparent cover comprises two transparent parts which form an included angle. One transparent part faces the upper side of the vehicle body 1, and the other transparent part faces the lateral side of the vehicle body 1 to enlarge the light emission angle of the indicator light source. In one example of the present invention, as shown in FIG. 8, there are two working indicators 67 disposed opposite each other on two sides of the vehicle body 1, facilitating users to see the working state of the disinfection vehicle from several perspectives.

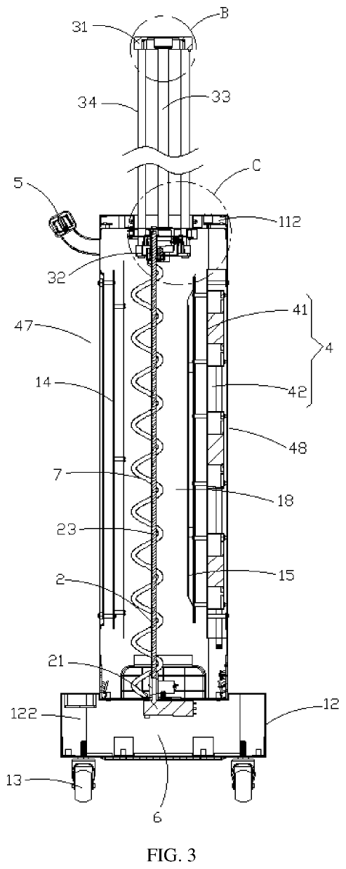

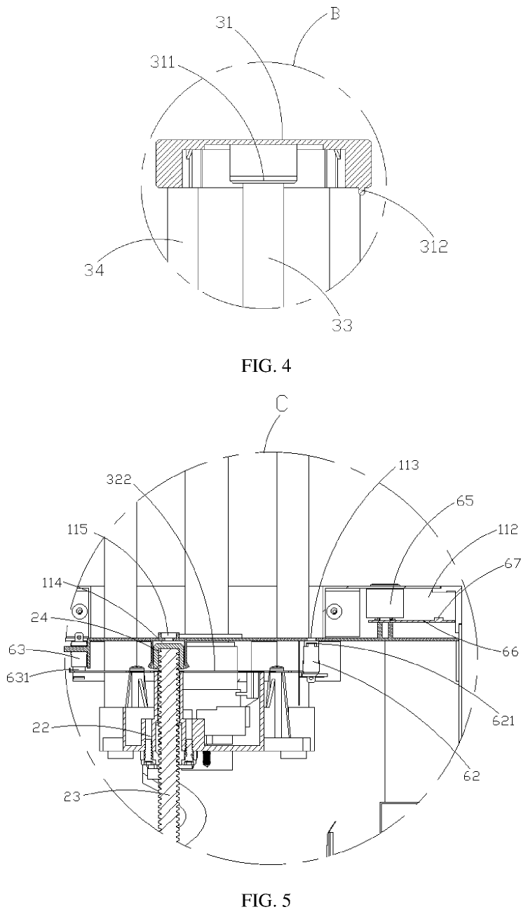

In this example, as shown in FIG. 4-5, the control system 6 also comprises the first limit switch 62, the second limit switch 63 and the master power supply 61. When the lifting mechanism 2 drives the disinfection component 3 to retract to the bottom of the vehicle body 1 and reach the limit state, the disinfection component 3 will enable the first limit switch 62 which disenables the lifting mechanism 2 via the master power supply 63 to make the disinfection component 3 keep the retracting state; when the lifting mechanism 2 drives the disinfection component 3 to stretch out of the vehicle body 1 and reach the limit state on the top, the disinfection component 3 will enable the second limit switch 62 which disenables the lifting mechanism 2 via the master power supply 63 to make the disinfection component 3 keep the stretching state.

Optionally, the first limit switch 62 is provided on the top of the first accommodating cavity 18 and has the first switch contact metal 621 facing the first fixing part 31 provided on the extrusion part 312; the vehicle body 1 is provided with the third avoiding hole 113 corresponding to the first switch contact metal 621, when the lifting mechanism 2 drives the disinfection component 3 to retract to the bottom of the vehicle body 1 and reach the limit state, the extrusion part 312 extends into the third avoiding hole 113 and goes against the first switch contact metal 621 to enable the first limit switch 62; the second limit switch 63 is provided on the top of the first accommodating cavity 18 and possesses a second switch contact metal 631 facing the second fixing part 32, when the lifting mechanism 2 drives the disinfection component 3 to stretch out of the vehicle body 1 and reach the limit state on the top of the vehicle body 1, the second fixing part 32 goes against the second switch contact metal 631 to enable the second limit switch 63.

As shown in FIG. 1 and FIG. 12, the lateral side close to the bottom of the vehicle body 1 is provided with a detection opening, the detection opening is provided with a mounting board 19 which can be assembled in the clamping way, facilitating repair and replacement of the sensing device 64. The sensing device 64 can be detected in the microwave way. The mounting board 19 can be made of plastic. The signal receiving face of the sensing device 64 faces the outer side of the vehicle body 1 and is inclined upward to increase the sensing area of the sensing device 64. When the disinfection component 3 is outside the vehicle body 1 for direct illumination disinfection, after the sensing device 64 finds human body, the control system 6 can automatically disenable the disinfection component 3, preventing people from being damaged by UV light emitted by the light source module 33. In one example of the present invention, the outline of the cross section of the vehicle body 1 is rectangular roughly, lateral sides of the vehicle body 1 in four directions are provides with a detection opening respectively, each detection opening is provided with a mounting board 19, each mounting board 19 is provided with a sensing device 64 to increase the detection scope of the sensing device 64.

In one example of the present invention, the mounting board 19 is provided with the first mounting columns 196 and two winding parts 191, the top of the first mounting column 196 is an upward inclined plane which facilitates installation of the sensing device 64 and enables the sensing side of the sensing device 64 to be upward inclined. There can be two first mounting columns 196. The top of one first mounting column 196 is provided with a protruding point, and the top of the other first mounting column 196 is provided with a fixed hole. The sensing device 64 is provided with two holes corresponding to two first mounting columns 196 respectively. One hole is used for working with the protruding point for positioning, and the other hole is used for fixing with a screw, so that the sensing device 64 can be installed and fixed quickly. The mounting board 19 is provided with two winding parts 191 which are in the hook shape. There is a certain clearance between winding parts 191 and the mounting board 19. The redundant lead wire 7 inside the vehicle body 1 can be wound on winding parts 191 to keep the tidiness inside the vehicle, optimize the space of the vehicle body 1 and avoid winding of wire during rotation of the lead screw 23.

In this example, the modular disinfection vehicle is also provided with the function of reminding the service life of the light source module 33 and the filter screen 45. One mounting board 19 is provided with a light source indicator 192, a light tube reset key 193, a filter screen indicator 194 and a filter screen reset key 195 electrically connecting to the master power supply 61. When the accumulated working time of the light source module 33 reaches the service life, the control system 6 will enable the light source indicator 192. After replacing a new light source module 33, by pressing the light tube reset key 193, the light source indicator 192 will be turned off, and the control system 6 will recalculate the service time of the light source module 33. After the service life of the filter screen 45 expires, the control system 6 will turn on the filter screen indicator 194. After replacing a new filter screen 45, by pressing the filter screen reset key 195, the filter screen indicator 194 will be turned off.

As shown in FIG. 3 and FIG. 6, the base 12 is provided under the vehicle body 1, there is a third accommodating cavity 122 inside the base 12, and the third accommodating cavity 122 connects to the first accommodating cavity 18. Optionally, the master power supply 61 and the motor 21 are provided in the third accommodating cavity 122, the power switch 69 of the master power supply 61 is provided on the lateral side of the base 12, the power cable of the master power supply 61 extends out of the base 12 from one side where the power switch 69 is located, the bottom of the base 12 is provided with a third ventilation hole 121 which connects to the third accommodating cavity 122, the base 12 also comprises a second fan 68 provided in the third accommodating cavity 122 corresponding to the master power supply 61, and the second fan 68 is used for inhaling air via the third ventilation hole 121 at the bottom of the vehicle body 1 to disperse heat of the master power supply 61. When the disinfection component 3 is located inside the vehicle body 1 for ventilation and disinfection, air in the third accommodating cavity 122 is blown by the second fan 68 into the first accommodating cavity 18 for sterilization and disinfection before it is exhausted out of the vehicle body 1.

As shown in FIG. 8 and FIG. 9, the vehicle body 1 is provided with a handle 5 which is on the upper end nearby the vehicle body 1, both outer sides of the vehicle body 1 are provided with an mounting hole respectively, the handle 5 comprises the first supporting arm 52, a handle 51 and the second supporting arm 53 which are connected in sequence. The end of the first supporting arm 52 and the end of the second supporting arm 53 are inserted into an mounting hole respectively and connects to the inner wall of the mounting hole in a fixed way. The first supporting arm 52 and the second supporting arm 53 are provided with an upward curve structure respectively nearby the handle 51.

Optionally, the handle 5 is parallel to the top of the vehicle body 1; optionally, the handle 5 is parallel to the lateral side of the vehicle body 1, which avoids inclination of the disinfection vehicle when the disinfection vehicle is put upside down, facilitates packing and guarantees stable transportation; In addition, such a handle design mode is more suitable for cable winding of the disinfection vehicle and can improve the convenience in use.

In one example of the present invention, the vehicle body 1 is provided with the first side wall and the second side wall which are opposite to each other, the mounting hole of the first side wall is the first mounting hole 54, the mounting hole of the second side wall is the second mounting hole 55, and to guarantee the stability when carrying or pushing the disinfection vehicle via the handle 5, the first mounting hole 54 and the second mounting hole 55 are provided on the same level.

As shown in FIG. 9, the first supporting arm 52 possesses the first bending section 522, the second bending section 523 and the first main section between the first bending section 522 and the second bending section 523; the second supporting arm 53 possesses the third bending section 532, the fourth bending section 533 and the second main section between the third bending section 532 and the fourth bending section 533. The bending direction of the first bending section 522 is the same as that of the second bending section 523, the bending direction of the third bending section 532 is the same as that of the fourth bending section 533; the first bending section 522 is used for extending into the first mounting hole 54 and connecting to the inner wall of the first mounting hole 54 in a fixed way, the third bending section 532 is used for extending into the second mounting hole 55 and connecting to the inner wall of the second mounting hole 55 in a fixed way. The first bending section 522 and the third bending section 532 are of a prism structure respectively, avoiding rotation of the first supporting arm 52 in the first mounting hole 54 and rotation of the second supporting arm 53 in the second mounting hole 55.

Optionally, the first bending section 522 and the third bending section 532 are of a prism structure, the outline of the cross section of the first bending section 522 and the third bending section 532 can be a rectangle, triangle and a shape composed of straight sides and arc lines.

Handle 51 comprises the first handle shell 511 and the second handle shell 512 which are snap-fitted with each other to form the fourth accommodating cavity in an enclosing way. The second bending section 523 and the fourth bending section 533 go deep into the fourth accommodating cavity where they are fixed. The first handle shell 511 is provided with several second mounting columns 513 which are arranged along the length of the handle 51. The top of the second mounting columns 513 is provided with screw holes. The second handle shell 512 is provided with several connecting holes corresponding to the second mounting column 513. The connecting holes of the second handle shell 512 face the inner side of the vehicle body 1 to form the concealed design and improve the appearance effect of the handle 5. Several connecting holes on the handle 51 can also prevent slide.

The handle 51 is between the first supporting arm 52 and the second supporting arm 53, and there is a large space between the first supporting arm 52 and the second supporting arm 53; When the disinfection vehicle is not working, the cable can be wound onto the handle 5, which prevents the cable from falling onto the ground disorderly and facilitates movement of the disinfection vehicle. The first main section of the first supporting arm 52 is provided with an upward first curve structure 521 nearby the handle 51, the second main section of the second supporting arm 53 is provided with a second curve structure 531 nearby the handle 51; The straight distance between the first curve structure 521 and the handle 51 is the same as that between the second curve structure 531 and the handle 51, so that the acting force of the handle 51 on the first supporting arm 52 is the same as or approximate to that on the second supporting arm 53, which facilitates holding of the handle 51. When winding the cable on the handle 5, the curve structure can also increase the winding space of the cable.

The above description only presents the preferred examples of the invention, and it is not for this reason that the patent scope of the invention is limited. Any equivalent structural transformation made by using the description of the invention and the drawings, or direct/indirect application in other related technical fields under the inventive concept of the invention, is included in the patent protection scope of the invention.

What is claimed is:

1. A modular disinfection vehicle which used for sterilization and disinfection of air or the object surface, the modular disinfection vehicle comprising:
a vehicle body with the first accommodating cavity and at least an avoiding hole connecting to the first accommodating cavity and provided on the top of the vehicle body;
a disinfection component comprising the first fixing part, the second fixing part, at least a support pillar and at least a light source module, the first fixing part and the second fixing part provided on each end of the support pillar respectively, the second fixing part provided in the first accommodating cavity, the light source module and the support pillar pass through the avoiding hole in a flexible way; the light source module installed on the first fixing part and/or the second fixing part in a dismountable way;
a lifting mechanism provided in the first accommodating cavity and connecting to the second fixing part to drive the disinfection component to stretch out of and retract to the vehicle body; and
a control system used for controlling enabling and disenabling of the light source module and controlling the lifting mechanism to drive the disinfection component to stretch out of and retract to the vehicle body in the vertical direction.

2. The modular disinfection vehicle as claimed in claim 1, wherein the first fixing part is provided with the first installation position, the second fixing part is provided with the second installation position, both ends of the light source module are installed at the first installation position and the second installation position respectively; the first fixing part and the support pillar are connected in a dismountable way, after the first fixing part and the support pillar are disassembled, aid light source module can be separated from the first installation position and the second installation position to finish disassembly and replacement of the light source module;
and/or, the second fixing part comprises the third shielding part provided on the top of the second fixing part for shielding the avoiding hole and preventing UV light from entering into the first accommodating cavity via the avoiding hole.

3. The modular disinfection vehicle as claimed in claim 1, wherein the outer side of the top of the vehicle body is provided with a groove used for accommodating the first fixing part, and the avoiding hole is provided at the bottom of the groove.

4. The modular disinfection vehicle as claimed in claim 3, wherein the horizontal projection plane of the first fixing part is rectangular on the whole;
and/or, the horizontal projection plane of the groove is rectangular on the whole;
and/or, there are four the support pillars provided on the outer side of the disinfection component;
and/or, the support pillar is a profile structure and the outer contour of its cross section is non-circular.

5. The multi-angle light source device as claimed in claim 4, wherein the connecting portion comprises a sleeve and an internal-hollow axis sleeved on the sleeve and rotated relative to the sleeve.

6. The modular disinfection vehicle as claimed in claim 1, wherein the vehicle body comprises a top cover provided on the top of the vehicle body and forming the second accommodating cavity with other components in an enclosing way, the second accommodating cavity is used for accommodating electronic components, the control system comprises the first circuit board provided in the second accommodating cavity;
the control system comprises a working indicator provided on the top of the vehicle body, the working indicator comprises an indicator light source provided on the first circuit board and a transparent cover provided corresponding to the indicator light source, the working indicator is used for displaying the working state of the light source module;
and/or, the first circuit board connects to at least one key used for controlling stretching or retraction of the lifting mechanism, controlling enabling or disenabling of the disinfection component or controlling the working time of the light source module.

7. The modular disinfection vehicle as claimed in claim 1, wherein the control system comprises the first limit switch, the second limit switch and the master power supply; when the lifting mechanism drives the disinfection component to retract to the bottom of the vehicle body and reach the limit state, the disinfection component starts the first limit switch which powers off the lifting mechanism via the master power supply to remain the retracting state of the disinfection component; when the lifting mechanism drives the disinfection component to stretch out of the vehicle body and reach the limit state on the top of the vehicle body, the disinfection component starts the second limit switch which powers off the lifting mechanism via the master power supply to remain the stretching state of the disinfection component.

8. The modular disinfection vehicle as claimed in claim 6, wherein the first limit switch is provided on the top of the first accommodating cavity, the first limit switch is provided with the first switch contact metal facing the first fixing part, the first fixing part is provided with an extrusion part, the vehicle body is provided with third avoiding hole corresponding to the first switch contact metal, when the lifting mechanism drives the disinfection component to retract to the bottom of the vehicle body and reach the limit state, the extrusion part stretch into the third avoiding hole to go against the first switch contact metal to start the first limit switch;
and/or, the second limit switch is provided on the top of the first accommodating cavity, the second limit switch is provided with the second switch contact metal facing the second fixing part, when the lifting mechanism drives the disinfection component to stretch out of the vehicle body and reach the limit state on the top of the vehicle body, the second fixing part goes against the second switch contact metal to start the second limit switch.

9. The modular disinfection vehicle as claimed in claim 1, wherein the modular disinfection vehicle also comprises a ventilation component provided in the first accommodating cavity, the vehicle body is provided with the first ventilation hole and the second ventilation hole connecting to the first accommodating cavity, the ventilation component is provided in the second ventilation hole;
air flows into the first accommodating cavity via the first ventilation hole, air is discharged out of the vehicle body via the second ventilation hole after sterilization and disinfection in the first accommodating cavity and then the vehicle body is provided with a shielding device corresponding to the first ventilation hole and the second ventilation hole respectively, the shielding devices are used for preventing UV light of the light source module in the first accommodating cavity from being emitted out of the vehicle body.

10. The modular disinfection vehicle as claimed in claim 8, wherein the shielding device is the first shielding part which comprises the first board and the second board provided at intervals, the first board is provided above the clearance of the second board in a stack-up way, disenabling UV light emitted by the light source module to be emitted out of the vehicle body in the direction vertical to the first board and the second board;
or one of the shielding devices is the second shielding part which comprises the fourth board and the fifth board, the middle part of the fourth board is provided with a ventilation hole, and the fifth board is provided at the ventilation hole of the fourth board at intervals in a stack-up way.

11. The modular disinfection vehicle as claimed in claim 8, wherein the first ventilation hole and the second ventilation hole are located on each side of the vehicle body, respectively;
and/or, the first ventilation hole is provided with a filter screen in a dismountable way;
or/and, both the first ventilation hole and the second ventilation hole are provided with a net shield;
or/and, the ventilation component comprises a fan housing and at least one first fan, the fan housing is provided with a fourth ventilation hole corresponding to the first fan, the fan housing is against the inner wall of the vehicle body, so that air in the first accommodating cavity can be exhausted out of the vehicle body via the second ventilation hole after passing through the fourth ventilation hole.

12. The modular disinfection vehicle as claimed in claim 1, wherein the lifting mechanism comprises a motor and supporting nuts and lead screws, the second fixing part is provided with the third through-hole in the vertical direction, the nuts are fixed inside the through-hole, the lead screw has the first end and the second end, the first end of the lead screw connects to the motor, the second end of the lead screw passes through the third through-hole and extends to the top of the first accommodating cavity; the vehicle body is provided with positioning holes, the second end of the lead screw is installed inside the positioning hole.

13. The modular disinfection vehicle as claimed in claim 11, wherein the second end of the lead screw is provided with a shaft sleeve provided in the positioning hole;
or/and, the motor is provided with a gear mechanism used for reducing the rotation speed of the motor;
or/and, the vehicle body is provided with an oil leakage hole corresponding to the positioning hole, and the oil leakage hole connects to the positioning hole.

14. The modular disinfection vehicle as claimed in claim 1, wherein the vehicle body is provided with a handle which is close to the upper end of the vehicle body; both outer sides of the vehicle body are provided with a mounting hole respectively, two mounting holes are opposite to each other, the handle comprises the first supporting arm, a handle and the second supporting arm which are connected in sequence, the end of the first supporting arm and the end of the second supporting arm are inserted in the mounting hole respectively and connect to the inner wall of the mounting hole.

15. The modular disinfection vehicle as claimed in claim 13, wherein the first supporting arm and the second supporting arm are respectively provided with an upward curve structure close to the handle;
and/or, the handle is parallel to and level with the top of the vehicle body;
and/or, the handle is parallel to and level with the lateral side of the vehicle body.

16. The modular disinfection vehicle as claimed in claim 1, wherein the vehicle body comprises a base provided with the third accommodating cavity connecting to the first accommodating cavity, the control system comprises a master power supply provided inside the third accommodating cavity; the bottom of the base is provided with the third ventilation hole connecting to the third accommodating cavity, the base also comprises the second fan of the third accommodating cavity, the second fan is provided corresponding to the master power supply for inhaling air via the third ventilation hole at the bottom of the vehicle body to disperse heat of the master power supply and blowing into the first accommodating cavity to finish sterilization and disinfection before the air is exhausted out of the vehicle body.

17. The modular disinfection vehicle as claimed in claim 1, wherein the vehicle body's lateral side close to the bottom is provided with a detection opening and a mounting board installed at the detection opening, the inner side of the mounting board is provided with a sensing device, the sensing side of the sensing device is provided upward, the sensing device connects to the control system electrically to control enabling or disenabling of the light source module.

18. The modular disinfection vehicle as claimed in claim 16, wherein the mounting board is provided with the first mounting column whose top is an inclined plane, the sensing device is against the inclined plane to realize that the sensing side of the sensing device is provided upward;
or/and, the inside of the mounting board is provided with a hook-like winding part.
